SEQUENCE LISTING (6 sequences)

SEQ ID NO: 1 - DNA, 24 bp, Artificial Sequence
  Synthetic primer
accaactgggacgacatggagaaa

SEQ ID NO: 2 - DNA, 23 bp, Artificial Sequence
  Synthetic primer
gggatagcacagcctggatagca

SEQ ID NO: 3 - DNA, 22 bp, Artificial Sequence
  Synthetic primer
gctttagcttggaagcaatgtt

SEQ ID NO: 4 - DNA, 20 bp, Artificial Sequence
  Synthetic primer
tcatacagctgccgtttcag

SEQ ID NO: 5 - DNA, 19 bp, Artificial Sequence
  Synthetic primer
ggcgcctcatgttctgcta

SEQ ID NO: 6 - DNA, 21 bp, Artificial Sequence
  Synthetic primer
gtgcgttggtcaccatgtaga

(19) United States
(12) Patent Application Publication
(10) Pub. No.: US 2019/0015503 A1
(43) Pub. Date: Jan. 17, 2019

(54) METHODS FOR THE PROGNOSIS AND TREATMENT OF ENDOMETRIAL CARCINOMA

(71) Applicants: INSERM (INSTITUT NATIONAL DE LA SANTÉ DE LA RECHERCHE MÉDICALE, Paris (FR); UNIVERSITÉ PARIS DESCARTES, Paris (FR); ASSISTANCE PUBLIQUE-HÔPITAUX DE PARIS (APHP), Paris (FR)

(72) Inventors: Patricia Forgez, Paris (FR); Anne Gompel, Paris (FR)

(21) Appl. No.: 15/745,211

(22) PCT Filed: Jul. 13, 2016
(86) PCT No.: PCT/EP2016/066693

§ 371 Date: Jan. 16, 2018

(30) Foreign Application Priority Data
Jul. 17, 2015 (EP) 15306170.0

(51) Int. Cl. A61K 39/395; A61P 35/00

(52) U.S. Cl. CPC A61K 39/395; C12Q 1/6886; A61P 35/00

(57) ABSTRACT

The present invention relates to methods for predicting the survival time of patients from endometrial adenocarcinoma. The present invention also relates to methods and pharmaceutical compositions for the treatment of endometrial adenocarcinoma.

### FIELD OF THE INVENTION

The present invention relates to methods for predicting the survival time of patients from endometrial adenocarcinoma. The present invention also relates to methods and pharmaceutical compositions for the treatment of endometrial adenocarcinoma

### BACKGROUND OF THE INVENTION

Endometrial cancer is, after breast cancer, the most frequent gynecologic cancer in high and intermediate income countries, and the fourth cancer in women after breast, colon and lung cancers [1]. About 75% of endometrial cancers are diagnosed at an early stage, but more than two thirds of patients with advanced stages will die from cancer, despite improvements in diagnosis, surgery, and radiotherapy [2-4]. The discovery and characterization of new markers remain necessary in order to evaluate more accurately the prognosis, and to develop specific treatments targeted to metastasis initiation and progression processes. This is sustained by the recent data from The Cancer Genome Atlas cohort (TCGA) reclassifying endometrial cancer in four categories for tailoring treatments [5, 6].

Neurotensin (NTS) and his high affinity NTS receptor 1 (NTSR1) have been reported to be implicated in carcinogenesis and tumoral progression in several cancers, as breast cancer [7, 8]. NTS is a 13 amino acids peptide formed from a precursor, cleaved by convertases. NTS is commonly known for its distribution along the gastrointestinal tract [9]. Typical physiological functions for NTS include stimulation of pancreatic and biliary secretions, inhibition of small bowel and gastric motility, and facilitation of fatty acids translocation [10-12]. The central and peripheral functions of NTS are mediated through its interactions with three receptors: NTSR1, NTSR2 and NTSR3 [13]. NTSR1 is poorly or not expressed in most tissues, but we previously demonstrated that its over-expression is an unfavorable prognostic factor, notably in breast and lung cancers [8, 14]. The intracellular localization of the receptor allows activation by autocrine and paracrine mechanisms [15], this activation being linked to the Wnt/beta-catenin pathway [8, 14]. Its ligand, NTS, is variably expressed in normal tissues. Its presence has been demonstrated in rate uterus, in the cow endometrium, and is also expressed in human normal myometrium as well as in leiomyomas [6-18]. Consequently, NTS was reported in functions linked to neoplastic progression, including cellular function related to tumor growth as proliferation and survival, as well as function enhancing the metastatic process, as invasion, and migration of the pancreas, prostate, colon, lung, and breast cancer cells [14, 19-21].

Exposure to unopposed endogenous estrogen is associated with an increased risk for developing endometrial cancer [22, 23]. Estradiol (E2) regulates NTS expression in central nervous system areas rich in ERα, as well as in normal epithelial breast cells [7, 8 24, 25]. Furthermore, administration of NTS and E2 enhances DNA synthesis in uterus [26], in which NTSR1 binding sites were also found [27]. NTS gene activation by E2 is mediated through a non-genomic pathway including the activation of cAMP/PKA/CREB and its binding on CRE (cAMP-responsive element) elements located in the proximal region of NTS promoter [28].

### SUMMARY OF THE INVENTION

The present invention relates to methods for predicting the survival time of patients from endometrial adenocarcinoma. The present invention also relates to methods and pharmaceutical compositions for the treatment of endometrial adenocarcinoma. In particular, the present invention is defined by the claims.

### DETAILED DESCRIPTION OF THE INVENTION

The inventors had analyzed NTS and NTSR1 expression and its prognostic value in a series of 100 cases of endometrial adenocarcinoma, in comparison with 66 cases of normal and hyperplasic endometrium samples, using immunochemistry and RT-PCR. Expression of NTS and NTSR1 in endometrium was demonstrated, using both immunohistochemistry and RT-PCR. NTSR1 was significantly overexpressed in endometrial adenocarcinoma versus nonmalignant endometrium (p<0.0001). NTS expression was significantly higher in endometrial adenocarcinoma than in normal endometrium (p<0.0001). Image-analysis based density evaluation of global staining for NTSR1 as well as the cytoplasmic semi-quantitative NTSR1 score were significantly associated with a worse histological grade (p<0.001) and were significantly associated with a reduction of overall survival (p=0.02 and p<0.01, respectively). In multivariate analysis, high global and cytoplasmic NTSR1 expression remained an independent marker of poor prognosis (p=0.004). Inter-observer reproducibility of cytoplasmic semi-quantitative score was high (intraclass correlation coefficient: 0.89 [CI95%: 0.86; 0.92]). The inventors provided evidence for NTS/NTSR1 as a contributor to endometrial cancer progression, through an upregulation of cytoplasmic NTSR1 in malignant tumors. They pointed out the interesting prognostic value of NTSR1 in its intra-cytoplasmic localization, this marker being an independent marker of poor survival. Showing a very good inter-observer reproducibility, NTSR1 could be easily used as an immunohistochemical marker to evaluate the prognosis. These findings support the therapeutic potential of NTS/NTSR1 inhibition or drug cellular targeting through NTSR1 in advanced stages of human endometrial cancers.

As used herein, the term “NTSR1” has its general meaning in the art and refers to neurotensin receptor 1 (Gene ID: 4923) which belongs to the large superfamily of G-protein coupled receptors. The natural ligand of NTSR1 is neurotensin (NTS).

Methods for Predicting the Survival Time:

One aspect of the present invention relates to a method for predicting the survival time of a subject suffering from an endometrial adenocarcinoma comprising i) determining the expression level of NTSR1 expression in a tumor tissue sample obtained from the subject, ii) comparing the expression level determined at step i) with its predetermined reference value and ii) concluding that the subject will have a long survival time when the expression level of NTSR1 is lower than its predetermined reference value or concluding that the subject will have a short survival time when the expression level of NTSR1 is higher than its predetermined reference value.

The method of the present invention is particularly suitable for predicting the duration of the overall survival (OS), progression-free survival (PFS) and/or the disease-free survival (DFS) of the subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

As used herein, the term “tumor tissue sample” has its general meaning in the art and encompasses pieces or slices of tissue that have been removed including following a surgical tumor resection or following the collection of a tissue sample for biopsy. The tumor tissue sample can be subjected to a variety of well-known post-collection preparative and storage techniques (e.g., fixation, storage, freezing, etc.) prior to determining the level of the NTSR1 expression. Typically the tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (using an IHC automate such as BenchMark® XT, for obtaining stained slides).

Measuring the expression level of NTSR1 can be performed by a variety of techniques well known in the art.

In some embodiments, the expression level is determined at nucleic acid level. Typically, the expression level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Puhlication No. 2003/0165951 as well as PCT Puhlication No. 99/26299 (puhlished May 27, 1999). Separate populations of semiconductor nanocrystals can he produced that are identifiable based on their different spectral characteristics. For example, semiconductor nanocrystals can he produced that emit light of different colors hased on their composition, size or size and composition. For example, quantum dots that emit light at different wavelengths based on size (565 mn, 655 nm, 705 mn, or 800 mn emission wavelengths), which are suitable as fluorescent labels in the probes disclosed herein are available from Life Technologies (Carlshad, Calif.).

Additional labels include, for example, radioisotopes (such as ³H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Puhlication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pre-treated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will he appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from and subjecting the RNAs to amplification and hybridization to specific probes, more particularly by means of a quantitative or semi-quantitative RT-PCR.

In some embodiments, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute expression level or normalized expression level. Typically, expression levels are normalized by correcting the absolute expression level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the expression level in one sample, e.g., a subject sample, to another sample, or between samples from different sources.

In some embodiments, the expression level of NTSR1 is determined at the protein level by any well-known method in the art.

Typically, such methods comprise contacting the tumor tissue sample with at least one selective binding agent capable of selectively interacting with NTSR1. The selective binding agent may be polyclonal antibody or monoclonal antibody, an antibody fragment, synthetic antibodies, or other protein-specific agents such as nucleic acid or peptide aptamers. Several antibodies have been described in the prior art and many antibodies are also commercially available such as described in the EXAMPLE.

For the detection of the antibody that makes the presence of the NTSR1 detectable by microscopy or an automated analysis system, the antibodies may be tagged directly with detectable labels such as enzymes, chromogens or fluorescent probes or indirectly detected with a secondary antibody conjugated with detectable labels. For example, one or more labels can be attached to the antibody, thereby permitting detection of the target protein (i.e NTSR1). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e g luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 125I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine.

In some embodiments, immunohistochemistry is performed. Immunohistochemistry (IHC) is a staining method based on enzymatic reactions using a binding partner, such as an antibody (e.g., monoclonal or polyclonal antibodies) or other binding partner, to detect the expression of the marker of interest (i.e. NTSR1). Typically, IHC protocols include detection systems that make the presence of the markers visible, to either the human eye or an automated scanning system, for qualitative or quantitative analyses. In a direct IHC assay, binding is determined directly upon binding of the binding partner (e.g., first antibody) to the tissue or biomarker due to the use of a labeled reagent. In such methods, generally a slide-mounted tissue sample is stained with a labeled binding reagent (e.g., labeled antibody) using common IHC techniques. Thus, in exemplary IHC methods provided herein, the antibody is modified to contain a moiety capable of being detected (as described above). In some embodiments, the antibody is conjugated to a small molecule, e.g., biotin, that is detected via a labeled binding partner or antibody. In some embodiments, the IHC method is based on staining with an antibody that is detected by enzymatic staining with horseradish peroxidase. For example, the antibody can be biotinylated and detected with avidin or streptavidin conjugated to detectable protein, such as streptavidin-horseradish peroxidase. In other examples, the antibody can be conjugated to detectable proteins that permit direct detection, such as, for example, conjugated to a fluorescent protein, bioluminescent protein or enzyme. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. Immunohistochemistry typically includes the following steps: i) fixing said tumor sample with formalin, ii) embedding said tumor sample in paraffin, iii) cutting said tumor sample into sections for staining, iv) incubating said sections with the binding partner specific for the marker of interest (i.e. NTSR1) v) rinsing said sections and optionally vi) incubating said section with a secondary antibody and vii) revealing the antigen-antibody complex with avidin-biotin-peroxidase complex. Accordingly, the tissue tumor sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems.

The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. NTSR1). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. No. 8,023,714; U.S. Pat. No. 7,257,268; U.S. Pat. No. 7,219,016; U.S. Pat. No. 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. NTSR1) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., NTSR1) present in the sample. Optical density or percentage area values can be given a scaled score, for example on an integer scale, for example, 0-10, 0-5, or 0-3. In particular examples, the amount of NTSR1 in a sample is classified on a scale of 0-3, e.g., 0, NTSR1+1, NTSR1+2, and NTSR1+3. The amount of NTSR1 present is relative to the percentage of NTSR1 pixels, that is, low percentages of NTSR1 pixels indicates a low level of NTSR1 whereas high percentages of NTSR1 pixels indicate high levels of NTSR1. Scores can correlated with percentages of NTSR1 positive pixels, such that the percentage area that is stained is scored as 0, NTSR1+1, NTSR1+2, and NTSR1+3, representing no staining, less than 10% staining, 10-25% staining or more than 25% staining respectively. For example, if the ratio (e.g., strong pixel stain to total stained area) is more than 25% the tumor tissue is scored as NTSR1+3, if the ratio is 10-25% of strong positive stain to total stain the tumor tissue is scored as NTSR1+2, if the ratio less than 10% of strong positive stain to total stain the tumor tissue is scored as NTSR1+1, and if the ratio of strong positive stain to total stain is 0 the tumor tissue is scored as 0. A score of 0 or NTSR1+1 indicates low levels of NTSR1 in the tested sample, whereas a score of NTSR1+2 or NTSR1+3 indicates higher levels of NTSR1 in the tested samples.

In some embodiments, the method of the present invention comprises the steps consisting in i) providing one or more immunostained slices of tissue section obtained by an automated slide-staining system by using a binding partner capable of selectively interacting with NTSR1 (e.g. an antibody as above descried), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

In some embodiments, the NTSR1 cytoplasmic expression level (i.e. the level of NTSR1 in the cytoplasm of the cells in contrast with the expression at the membrane) is determined. In some embodiments, the NTSR1 global expression level (i.e. the level of NTSR1 expressed by cells wherever the localization of the marker (cytoplasm or membrane)) is determined.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of NTSR1 in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of NTSR1 in a group of reference, one can use algorithmic analysis for the statistic treatment of the measured expression levels of NTSR1 in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

A predetermined reference value can be relative to a number or value derived from population studies, including without limitation, subjects of the same or similar age range, subjects in the same or similar ethnic group, and subjects having the same severity of cancer. Such predetermined reference values can be derived from statistical analyses and/or risk prediction data of populations obtained from mathematical algorithms and computed indices. In some embodiments, the predetermined reference values are derived from the expression level of NTSR1 in a control sample derived from one or more subjects who do not suffer from cancer. Furthermore, retrospective measurement of the expression level of NTSR1 in properly banked historical subject samples may be used in establishing these predetermined reference values.

In some embodiments, the predetermined reference value is determined by carrying out a method comprising the steps of

a) providing a collection of tumor samples from subject suffering from the same cancer;

b) providing, for each tumor sample provided at step a), information relating to the actual clinical outcome for the corresponding subject (i.e. the duration of the disease-free survival (DFS) and/or the overall survival (OS));

c) providing a serial of arbitrary quantification values;

d) determining the level of NTSR1 for each tumor sample contained in the collection provided at step a);

e) classifying said tumor samples in two groups for one specific arbitrary quantification value provided at step c), respectively: (i) a first group comprising tumor samples that exhibit a quantification value for level that is lower than the said arbitrary quantification value contained in the said serial of quantification values; (ii) a second group comprising tumor samples that exhibit a quantification value for said level that is higher than the said arbitrary quantification value contained in the said serial of quantification values; whereby two groups of tumor samples are obtained for the said specific quantification value, wherein the tumor samples of each group are separately enumerated;

f) calculating the statistical significance between (i) the quantification value obtained at step e) and (ii) the actual clinical outcome of the subjects from which tumor samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant) has been calculated at step g).

For example the expression level of NTSR1 has been assessed for 100 tumor samples of 100 subjects. The 100 samples are ranked according to the expression level of NTSR1. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of NTSR1 corresponding to the boundary between both subsets for which the p value is minimum is considered as the predetermined reference value. It should be noted that the predetermined reference value is not necessarily the median value of levels of NTSR1. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level of NTSR1 with the range of values which are identified. In certain embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a subject may be assessed by comparing values obtained by measuring the expression level of NTSR1, where values greater than 5 reveal a poor prognosis and values less than 5 reveal a good prognosis. In a another embodiment, a subject may be assessed by comparing values obtained by measuring the expression level of NTSR1 and comparing the values on a scale, where values above the range of 4-6 indicate a poor prognosis and values below the range of 4-6 indicate a good prognosis, with values falling within the range of 4-6 indicating an intermediate occurrence (or prognosis).

Methods of Treatment:

One aspect of the present invention relates to a method of treating endometrial cancer in a subject in need thereof comprising administering to the subject a therapeutically effective amount of an inhibitor of NTSR1 activation or expression.

As used herein, the term “inhibitor of NTSR1 activation or expression” should be understood broadly, this expression refers to agents down-regulating the expression of neurotensin or of neurotensin receptor 1, compounds that bind to neurotensin (NTS) or NTSR1 and inhibit the neurotensin activation of NTSR1, or a protease that can degrade NTS.

Examples of inhibitors of NTSR1 activation or expression may be selected from the group consisting of an agent down-regulating the expression of NTS or NTSR1, an antibody against NTS or a fragment thereof which binds to NTS, an antibody against the NTSR1 or a fragment thereof which binds to the NTSR1, and an antagonist of the NTSR1.

As used herein, the term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In some embodiments, the antibody is a “chimeric” antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

In some embodiments, the antibody is a humanized antibody. “Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

In some embodiments, the antibody is a human antibody. A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e g, immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

In some embodiments, the antibody is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the antibody is an anti-NTSR1 monoclonal antibody-drug conjugate. An “anti-NTSR1 monoclonal antibody-drug conjugate” as used herein refers to an anti-NTSR1 monoclonal antibody according to the invention conjugated to a therapeutic agent. Such anti-NTSR1 monoclonal antibody-drug conjugates produce clinically beneficial effects on NTSR1-expressing tumor cells when administered to a subject. In typical embodiments, an anti-NTSR1 monoclonal antibody is conjugated to a cytotoxic agent, such that the resulting antibody-drug conjugate exerts a cytotoxic or cytostatic effect on a NTSR1-expressing tumor cell when taken up or internalized by the cell. Any cytotoxic agent well known by the skilled person may use. In some embodiments, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP (dimethylvaline-valine-dolaisoleuine-dolaproine-phenylalanine-p-phenylenediamine), MMAF (dovaline-valine-dolaisoleunine-dolaproine-phenylalanine), and MAE (monomethyl auristatin E). The synthesis and structure of auristatin E and its derivatives are described in U.S. Patent Application Pu blication No. 20030083263; International Patent Publication Nos. WO 2002/088172 and WO 2004/010957; and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In some embodiments, the anti-NTSR1 monoclonal antibody of the invention is used to induce antibody dependent cellular cytotoxicity (ADCC) or complement dependent cytotoxicity (CDC) against NTSR1-expressing cells. In some embodiments, the anti-NTSR1 antibody may be suitable for disturbing the expression of NTSR1 at the cell surface (e.g. by provoking internalization of NTSR1) so that cell migration, cell proliferation and tumour growth of tumor cells will be limited or inhibited. In some embodiments, an anti-NTSR1 monoclonal antibody of the invention is used to induce antibody dependent cellular cytotoxicity (ADCC). In ADCC, monoclonal antibodies bind to a target cell (e.g., cancer cell) and specific effector cells expressing receptors for the monoclonal antibody (e.g., NK cells, CD8+ T cells, monocytes, granulocytes) bind the monoclonal antibody/target cell complex resulting in target cell death. Accordingly, in some embodiments, an anti-NTSR1 monoclonal antibody comprising an Fc region with effector function is used to induce antibody dependent cellular cytotoxicity (ADCC) or complement dependent cytotoxicity (CDC) against a NTSR1-expressing cell. Methods for inducing ADCC generally include contacting the NTSR1-expressing cell with an effective amount an anti-NTSR1 monoclonal antibody comprising an Fc region having ADCC activity, wherein the contacting step is in the presence of a cytolytic immune effector cell expressing an Fc receptor having cytolytic activity. Immune effector cells expressing cytolytic Fc receptors (e.g., FcγRIIIα or CD16) include, for example, NK cells as well certain CD8+ T cells. Methods for inducing CDC generally include contacting the NTSR1-expressing cell with an effective amount an anti-NTSR1 monoclonal antibody comprising an Fc region having CDC activity, wherein the contacting step is in the presence of complement.

In some embodiments, the anti-NTSR1 antibody is monospecific, bispecific, trispecific, or of greater multispecificity. Multispecific antibodies, including bispecific and trispecific antibodies, useful for practicing the methods described herein are antibodies that immunospecifically bind to both NTSR1 and a second cell surface receptor or receptor complex that mediates ADCC, phagocytosis, and/or CDC, such as CD16/FcgRIII, CD64/FcgRI, killer inhibitory or activating receptors, or the complement control protein CD59. In a typical embodiment, the binding of the portion of the multispecific antibody to the second cell surface molecule or receptor complex enhances the effector functions of the anti-NTSR1 antibody. In some embodiment, the anti-NTSR1 antibody is a bispecific antibody. The term “bispecific antibody” has its general meaning in the art and refers to any molecule consisting of one binding site for a target antigen on tumor cells (i.e. a NTSR1 receptor) and a second binding side for an activating trigger molecule on an effector cell, such as CD3 on T-cells, CD16 (FcyR111) on natural killer (NK) cells, monocytes and macrophages, CD89 (FcαRI) and CD64 (FcyRI) on neutrophils and monocytes/macrophages, and DEC-205 on dendritic cells. According to the invention, the bispecific antibody comprises a binding site for NTSR1. tApart from the specific recruitment of the preferred effector cell population, bispecific antibodies avoid competition with endogenous immunoglobulin G (IgG) when the selected binding site for the trigger molecule on the effector cell does not overlap with Fc-binding epitopes. In addition, the use of single-chain Fv fragments instead of full-length immunoglobulin prevents the molecules from binding to Fc-receptors on non-cytotoxic cells, such as FcyRII on platelets and B-cells, to Fc-receptors that do not activate cytotoxic cells, including FcyRlllb on polymorphonuclear leukocytes (PMN), and to inhibitory Fc-receptors, such as FcyRllb on monocytes/macrophages. Methods for making bispecific antibodies are known in the art. Traditional production of full-length bispecific antibodies is based on the coexpression of two immunoglobulin heavy chain-light chain pairs, where the two chains have different specificities (see, e.g., Milstein et al., 1983, Nature 305:537-39). Because of the random assortment of immunoglobulin heavy and light chains, these hybridomas (quadromas) produce a potential mixture of 10 different antibody molecules, of which only one has the correct bispecific structure. Similar procedures are disclosed in International Publication No. WO 93/08829, and in Traunecker et al., 1991, EMBO J. 10:3655-59. Other examples of bispecific antibodies include Bi-specific T-cell engagers (BiTEs) that are a class of artificial bispecific monoclonal antibodies. BiTEs are fusion proteins consisting of two single-chain variable fragments (scFvs) of different antibodies, or amino acid sequences from four different genes, on a single peptide chain of about 55 kilodaltons. One of the scFvs binds to tumor antigen (i.e. NTSR1) and the other generally to the effector cell (e.g. a T cell via the CD3 receptor. Other bispecific antibodies those described in WO2006064136. In particular the bispecific antibody is a Fab format described in WO2006064136 comprising one VH or VHH specific for NTSR1 and one VH or VHH specific for an effector cell.

An “inhibitor of gene expression” refers to a natural or synthetic compound that has a biological effect to inhibit or significantly reduce the expression of a gene.

Inhibitors of gene expression for use in the present invention may be based on anti-sense oligonucleotide constructs. Anti-sense oligonucleotides, including anti-sense RNA molecules and anti-sense DNA molecules, would act to directly block the translation of the mRNA by binding thereto and thus preventing protein translation or increasing mRNA degradation, thus decreasing the level of the protein (e.g. NTSR1), and thus activity, in a cell. For example, antisense oligonucleotides of at least about 15 bases and complementary to unique regions of the mRNA transcript sequence encoding the targeted protein (e.g. NTSR1) can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting a subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the targeted proteins (e.g. NTSR1). Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

By a “therapeutically effective amount” is meant a sufficient amount of the inhibitor of NTSR1 activity or expression at a reasonable benefit/risk ratio applicable to the medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The inhibitor of NTSR1 activity or expression is typically combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The active ingredient can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. Survival analyses with NTSR1 mRNA z-score. Kaplan-Meier curves with log-rank test (A) overall survival (OS), (B) progression-free survival (PFS), (threshold: 90^{th} percentile).

FIG. 2. Immunohistochemical staining for NTSR1. (A) NTSR1 global staining determined by image analysis, sorted by benign and malignant samples. (B) Correlation between NTSR1 global staining and grade. (B) Correlation between NTSR1 cytoplasmic semi-quantitative score and grade. (C) Correlation between NTSR1 apical semi-quantitative score and grade. Kruskal-Wallis test with Duns post-analysis (*: p<0.05; **: p<0.01).

FIG. 3. Survival analyses. Kaplan-Meier curves with log-rank test for overall survival (OS) and progression-free survival (PFS). (A) and (B) WHO histological grade. (C) and (D) NTSR1 immunohistochemical semi-quantitative cytoplasmic score (threshold: 95^{th} percentile; score: 130). (E) and (F) NTSR1 immunohistochemical global staining (image analysis) (threshold: 95^{th} percentile).

FIG. 4. Immunohistochemical staining for long fragment NTS (LF NTS) and NTS peptide. (A) LF NTS and (B) NTS peptide, sorted by benign and malignant samples. Kruskal-Wallis test with Duns post-analysis (*: p<0.05; **: p<0.01).

FIG. 5. Neurotensin and NTSR1 mRNA transcripts. RT-PCR for NTS and NTSR1 mRNA transcripts in endometrial adenocarcinoma (lanes 1-10), normal endometrium (lanes N1 and N2), positive controls (lanes CL1 and CL2, A2780 and SKOV3 cell lines, respectively), and negative control (lane 0).

FIG. 6. Analysis of NTSR1 promoter methylation, from the uterine corpus endometrial carcinoma (UCEC) cohort of TCGA (HM450). (A) Correlation between NTSR1 mRNA z-score (RNASeq V2) and NTSR1 promoter methylation (beta-value >0.20); U-Mann Whitney test, Bonferroni correction for multiple comparisons (*: p<0.05; **: p<0.005). (B) Correlation between NTSR1 CpG 1, 5 and 11 and histological FIGO/WHO grade (Chi-square test).

### EXAMPLE

Material & Methods

Analyses of TCGA Data

Data from The Cancer Genome Atlas (TCGA) cohort of uterine corpus endometrial carcinoma (UCEC), initially published in Nature 5, were retrieved using cBioPortal [29, 30], including clinical data, main molecular features, NTSR1 mRNA z-scores (RNASeqV2) and NTSR1 copy number alterations, available in 333 cases. The methylation status of NTSR1 gene was analyzed in 221 cases of the TCGA provisional UCEC cohort, using the Illumina Human Methylation 450 (HM450) assay. A methylated status was defined by a beta-value greater than 0.20. The comparison of NTSR1 z-scores between clinicopathological and molecular groups were performed using the U-Mann Whitney test (two groups) or Kruskal-Wallis test with Duns post-analysis (more than two groups). The Spearman correlation coefficients were calculated to evaluate the correlation with other mRNA z-scores. Survival functions were performed with univariate and multivariate proportion hazard test Cox model (SAS 9.3 software, USA), and with the Log-rank test.

Population, Clinical Data, and Tissues

One hundred consecutive cases of endometrial adenocarcinoma were retrospectively retrieved from the files of the Department of Pathology (CHU de Nancy, France), including 89 endometrioid adenocarcinoma, 6 serous adenocarcinoma, 3 clear cell adenocarcinoma, and 2 mucinous adenocarcinoma, from 1 Jan. 2000 to 30 Jun. 2008 (Table 1). Sixty six nonmalignant endometrial samples were also retrieved, including normal endometrial tissues (n=17), endometrial polyps (n=14), and cases of simple (n=13), complex (n=9) and atypical (n=13) hyperplasia.

The histological diagnosis was checked by microscopic examination of sections stained with hematoxylin, eosin and saffron. In cancer, histological subtype, grade according to the criteria of the WHO classification [31], myometrial invasion, neoplastic emboli, local and nodal invasion, were reviewed by two experimented pathologists. A representative paraffin block from each case was selected. When several were available, the block with the higher tumor cell density was chosen.

Main clinical data were retrospectively collected from the Department of Gynecological Surgery (CHU de Nancy, France) and the Department of Radiotherapy (Institut de Cancérologie de Lorraine, France), including age, diagnostic circumstances, body mass index, medical history, menarche and menopause age, hormonal treatment, cancer treatments, follow-up data. TNM and FIGO stages were determinated [32].

Ethics

The experiments reported here were carried out under the current French ethical regulations as study was carried out according to the Declaration of Helsinki principles and in agreement with the French laws on biomedical research (institutional review board no DC2008-459; CNIL declaration no 1209171). Anonymity of patients was strictly respected, following local ethical guidelines.

Immunohistochemistry

Five μm paraffin sections were immersed in a 10 mM sodium citrate buffer (pH 6) for 20 minutes at 97° C. for dewaxing and antigen retrieval. Staining for NTSR1 was evaluated with a primary anti-NTSR1 antibody (1/100; goat polyclonal sc-7596, Santa Cruz, USA). Additionally, staining for NTS was evaluated in 18 samples of cancer, 20 samples of hyperplasia, and 10 samples of normal endometrium, using an antibody aiming NTS precursor (1/50 overnight; mouse monoclonal, homemade antibody) and NTS peptide (1/50 overnight; mouse monoclonal, homemade antibody). Optimized dilutions were previously established on 10 tissue endometrial specimens by performing serial dilution of antibodies. Immunohistochemistry was performed with Dako Autostainer Plus (Dako Cytomation, Glostrup, Denmark) and Flex+ Envision revelation system (Dako). Negative controls were used throughout the experiment.

Cytoplasmic and membranous staining for NTSR1 were evaluated using a semi-quantitative score. The staining intensity was graded as following: 1, weak; 2, medium; and 3, strong. A score was obtained by multiplying the percentage of positive cells by the intensity level. A cytoplasmic score and a membranous score between 0 and 300 were so obtained. Staining was performed independently by two observers (G.G. and M.A.), blinded to the conditions and clinical data. A mean value was then calculated for both scores.

Additionally, global staining for NTSR1 was evaluated using a semi-automatic image analysis method with Adobe Photoshop CS2 9.0 (Adobe Systems Incorporated, USA) and Image J 1.42u (Wayne Rasband, National Institutes of Health, USA). For each case, acquisition was performed with DP72 Olympus camera at ×100 magnification. Then, labeled areas were selected and copied in new JPEG files, using the Adobe Photoshop Color Range selection tool. After conversion into grayscale pictures and color inversion, integrated density was measured with ImageJ. Staining for NTS was evaluated using the same method.

RNA Extraction and RT-PCR

Frozen tumor tissues from 10 endometrial adenocarcinoma, and 2 normal endometrium cases were macrodissected. Total RNA was extracted from approximately 100 mg tissue after homogenization, using the Trizol kit (Invitrogen, Carlsbad, Calif., USA) according to the manufacturer's recommendations. Reverse transcription (RT) reactions were carried out under the following cycling conditions: 65° C. for 10 min and 37° C. for 1 hour, using First-Strand cDNA Synthesis Kit (GE Health Care, UK). Polymerase chain reaction (PCR) was conducted on a Rotor-Gene RG-3000 (Corbett Research, Sidney, Australia), according to standard protocols. The reaction mixture contained 1 μg of total cDNA from each sample, n μM of each primer (n=1 for NTS and n=0.05 for NTSR1 and Actin), 10 μl of 2× Absolute Blue QPCR SYBr Green Mix Plus ROX Vial (Thermo Scientific, UK) and water to a final volume of 20 μl. Positive and negative control samples were run for each RT-PCR assay. Reactions were carried out under the following cycling conditions: 95° C. for 15 min, and 40 cycles of 95° C. for 30 s, followed by 56° C. (for NTS) and 58° C. (for NTSR1 and Actin) for 30 s and 72° C. for 30 s, with a final primer sequence extension incubation of 72° C. for 15 min. Gene-specific primer pairs were located on two adjacent exons to achieve a high level of specificity and to avoid detection of genomic DNA (Table 2) [33,34]. PCR products were verified by assay with agarose gel electrophoresis (High Resolution Agarose, Eurobio). PCR products (10 μL each) were separated on 2% agarose gels in Tris-borate EDTA buffer (10×, Euromedex), stained with ethidium bromide, photographed under UV light. A 50-bp DNA Ladder (Promega Corporation) was used as a molecular size marker.

Statistical Analysis

Descriptive analyses were performed on the whole sample. Pearson's chi-square test to compare categorical variables and the ANOVA F statistic for quantitative variables were used. A p value of 0.05 was accepted as significant. In order to test the reliability between the two raters for both measures of cytoplasmic and membranous NTSR1, intraclass correlation coefficient (ICC) was used. Survival functions were performed with univariate and multivariate proportion hazard test Cox model, and with Kaplan-Meier method. Log-rank test were used to test the difference regarding survival among two groups, with a 95^{th} percentile cutoff. These statistical analyses were conducted using the SAS 9.3 software.

Results:

Clinical Data

Prognostic Value of NTSR1 mRNA in the TCGA Database

In the TCGA multicenter UCEC cohort 5, 6, among 333 endometrial adenocarcinoma cases, 271 (81.4%) were endometrioid-type, 52 (15.6%) serous-type and 10 (3%) mixt-type. Mean age of the patients was 63 years. 226 tumors were FIGO stage I (68.5%), 19 stage II (5.8%), 69 stage III (20.9%), and 16 stage IV (4.8%). Histological grades were as follows: 78 (23.4%) cases of grade 1, 91 (27.3%) grade 2 and 164 (49.3%) grade 3.

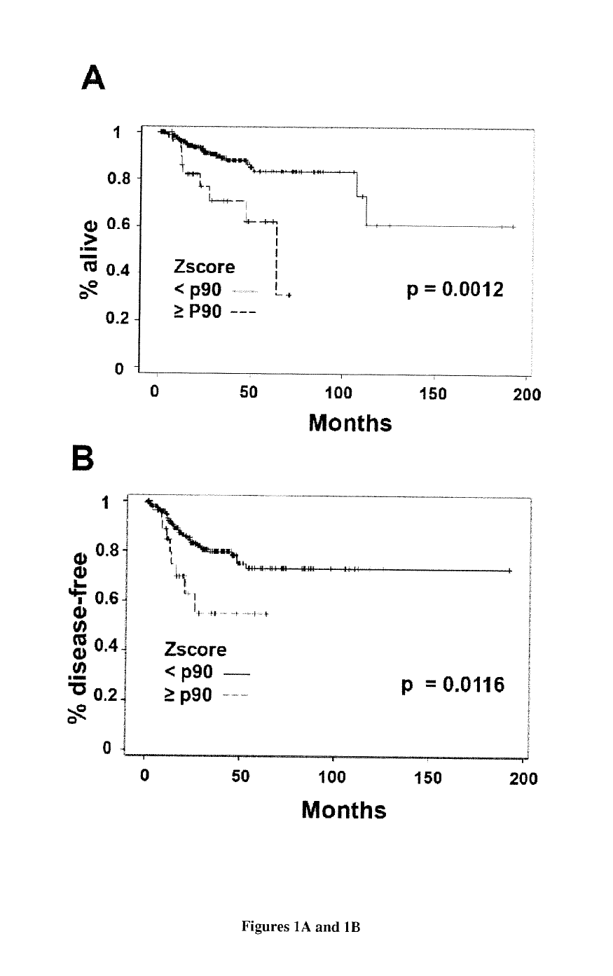

NTSR1 mRNA expression was significantly increased in higher grade tumors (p=0.0008). Using the Log-rank test (FIG. 1), NTSR1 mRNA level above the 90th percentile was associated with a significantly worse OS (log-rank: p=0.0012) and worse PFS (log-rank: p=0.0116). With the same threshold, and when including only endometrioid carcinomas, NTSR1 mRNA level continued to be negatively correlated with OS (log-rank: p<0.0001) and PFS (log-rank: p=0.0016).

Using the Cox model, a high NTSR1 mRNA z-score was significantly associated with a shorter OS and PFS, both in univariate (OS: p=0.0005 and PFS: p=0.0382) and multivariate Cox analyses (OS: p=0.0154; PFS: p=0.0048), and independently from the histological grade. After exclusion of the serous and mixed subgroups (endometrioid carcinoma only), NTSR1 mRNA level was still negatively correlated with OS (univariate: p=0.0001; multivariate: p=0.0019) and PFS (univariate: p=0.0005; multivariate: p=0.37) (data not shown).

NTSR1 Protein Expression and Localization in Benign Endometrium and Endometrial Adenocarcinoma

The clinical and the histological characteristics of the endometrial carcinoma cases obtained from the Nancy CHRU cohort are shown in table 1. The patients presented the classical risk factors for endometrial carcinoma: obesity (41%), overweight (21%), hypertension (53%) or diabetes mellitus (20%). The large majority of patients were post-menopausal (92%). Diagnostic circumstances included post-menopausal bleeding (81%), premenopausal bleeding (5%), and pelvic pain (4%). The predominant pathological subtype was endometrioid differentiation (89%). A positive node status was present in 6.8% of cases. The majority of the tumors were FIGO stage Ia (56%), and 11% of patients were metastatic. Thirteen (13%) women died from their disease related to the endometrial carcinoma.

NTSR1 score was also analyzed in normal endometrium and benign endometrial lesions. Seventy-two percent of the cases were positively labeled with the NTSR1 antibody in nonmalignant samples (48/66), showing a weak staining in most of positive samples. In the normal endometrium, irrespective of the phases of the menstrual cycle, NTSR1 staining was negative or very weak. In simple hyperplasia, NTSR1 was mildly positive with a membranous and cytoplasmic localization. The hyperplasia samples and polyps displayed equivalent staining.

In malignant tumors, NTSR1 distribution was in most of cases heterogeneous. Ninety percent of the cases were positively labeled with the NTSR1 antibody. In low and intermediate grade tumors, NTSR1 staining was frequently membranous and most notably apical, in association with a mild-to-moderate cytoplasm staining. NTSR1 cytoplasmic localization was predominant in higher grade tumors. For each specimen, cytoplasmic NTSR1 was semi-quantitatively scored, showing a very good inter-observer reproducibility (ICC: 0.8909 [C195%: 0.8553; 0.9181]).

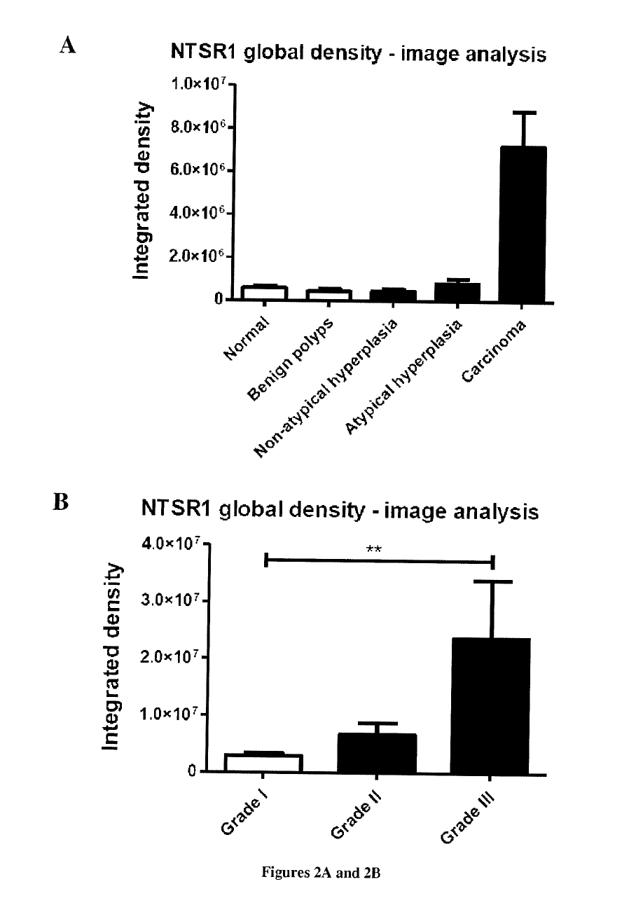

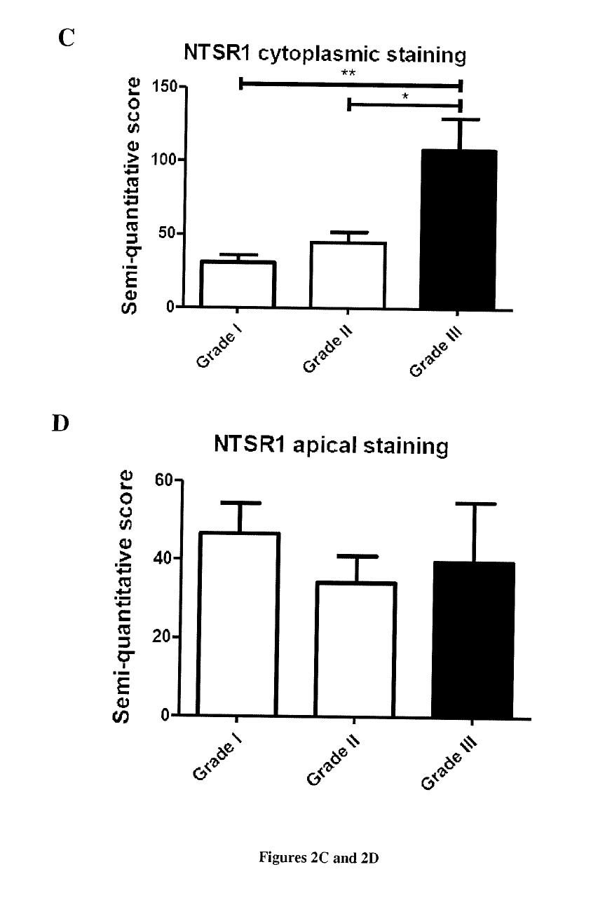

Immunohistochemistry showed a significant overexpression of NTSR1 in cancer samples, compared to benign tissues, with a global staining (image analysis-based standardized value) nearly 14-fold higher in endometrial adenocarcinoma (p<0.001) (Table 3). Cytoplasmic NTSR1 semi-quantitative score was significantly increased in endometrial adenocarcinoma vs. benign endometrium (p<0.001), without significant difference between the different benign samples (FIG. 2). Low NTSR1 expression was seen in atypical or non-atypical hyperplasia, with again a significantly higher NTSR1 expression in cancerous tissues as compared to hyperplasia (p=0.01). The global staining standardized value (image analysis) for NTSR1 showed a 6-fold significant increase in grade 3 as compared to grade 1 (p=0.01), associated with a significant increase of cytoplasmic semi-quantitative score in grade 3 vs. grade 1 (p=0.004) (FIG. 2). No significant differences were seen within the carcinoma histological subtypes. When regarding the apical score, no significant differences between the different tumor grades was found (p=0.43).

Survival Analyses

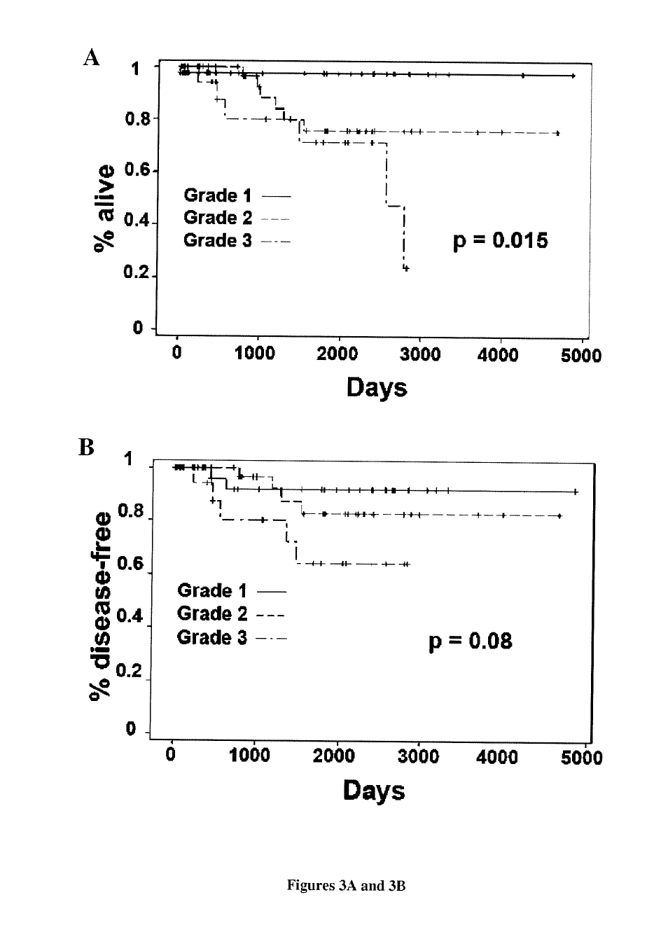

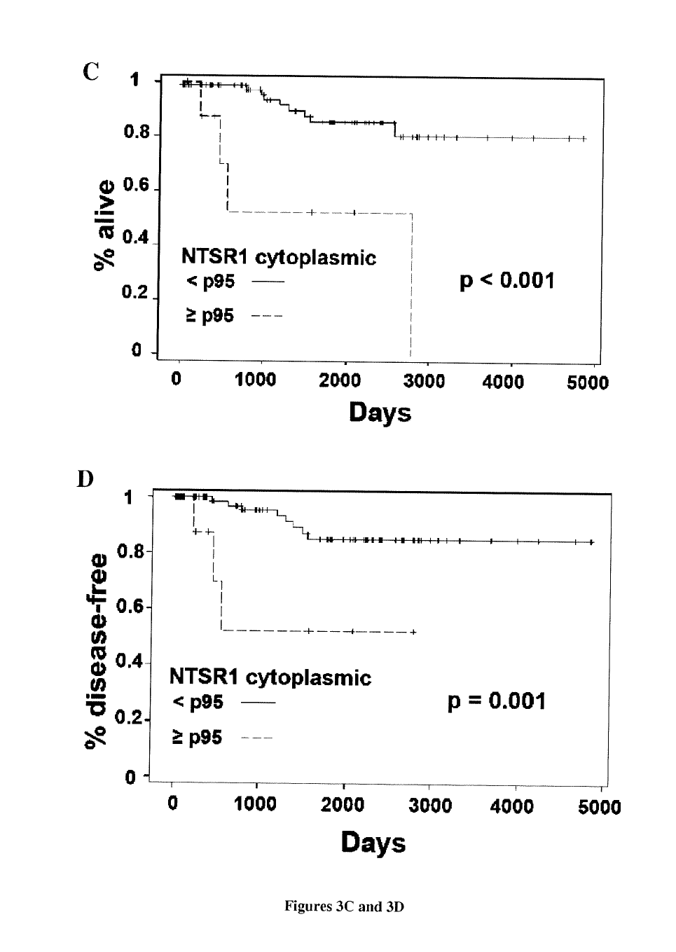

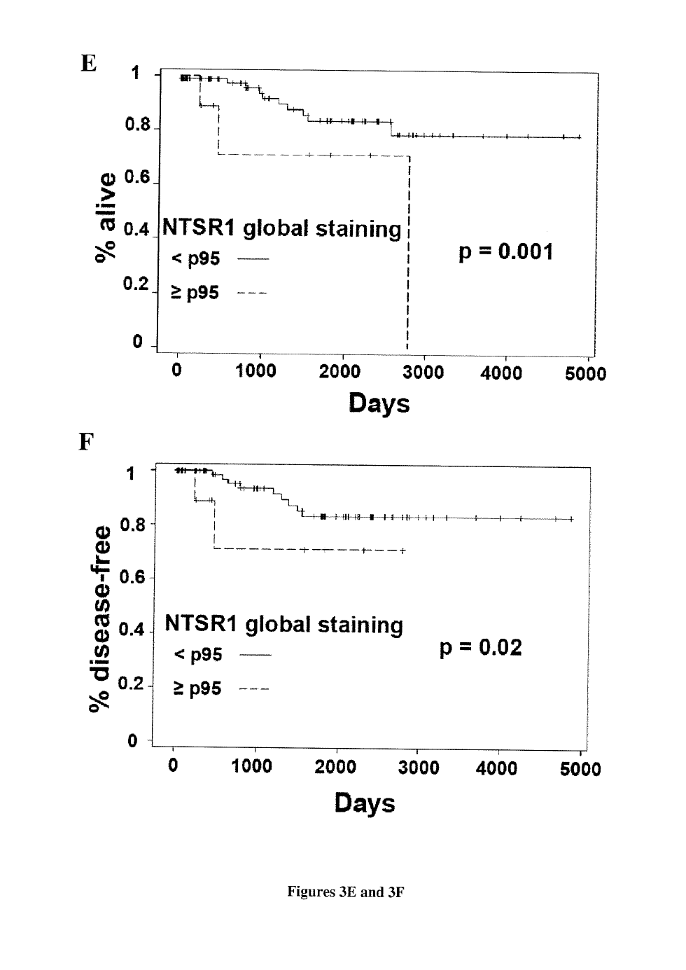

Follow up and overall 5-year survival data were available for 97% and 77% of the patients, respectively. Overall survival (OS) was significantly correlated with vaginal extension (p=0.0025) and the metastatic status (p<0.0001). A higher histological grade was correlated with a worse OS (p=0.015), but not progression-free survival (PFS) (p=0.08) (Log-rank test) (FIG. 3).

Using the univariate Cox model, NTSR1 cytoplasmic staining, as well as global staining standardized value, were negatively correlated with OS (p=0.02 and p=0.009, respectively). This correlation remained significant when excluding non endometrioid subtypes (p=0.04 and p=0.02, respectively). In multivariate analysis with stepwise selection, high NTSR1 expression remained an independent prognosis marker, negatively correlated with OS for both cytoplasmic semi-quantitative score (p=0.004) and global staining standardized value (p=0.0035). The presence of metastases was also an independent poor prognosis marker (p<0.001, OS). The cytoplasmic semi-quantitative score was negatively correlated with PFS (p=0.03) in multivariate analysis.

Using the Log-rank test (FIG. 3), a cytoplasmic NTSR1 expression score above the 95^{th} percentile (score greater than 130) was associated with a significantly worse OS (log-rank: p<0.001; 5-year OS=40% for a cytoplasmic score ≥130 vs. OS=83% if a score<130). With the same threshold, NTSR1 overexpression was also correlated with PFS (cytoplasmic semi-quantitative score: p=0.001; global staining standardized value: p=0.02).

NTS Expression

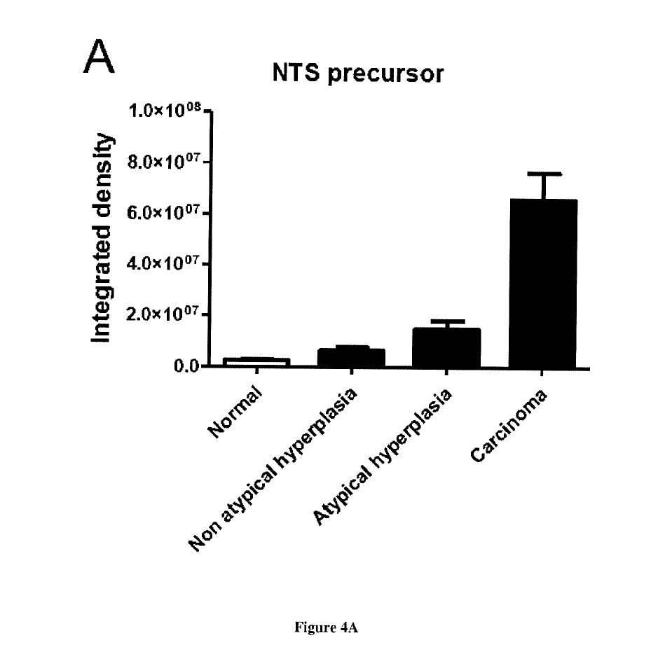

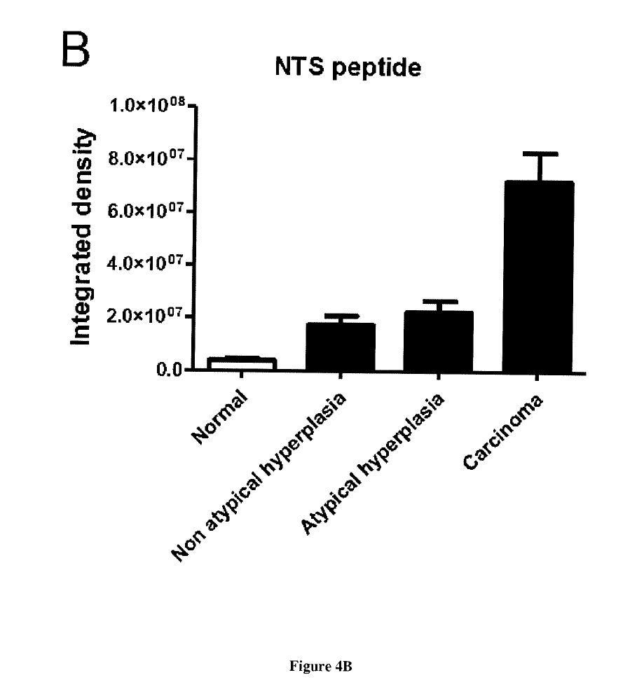

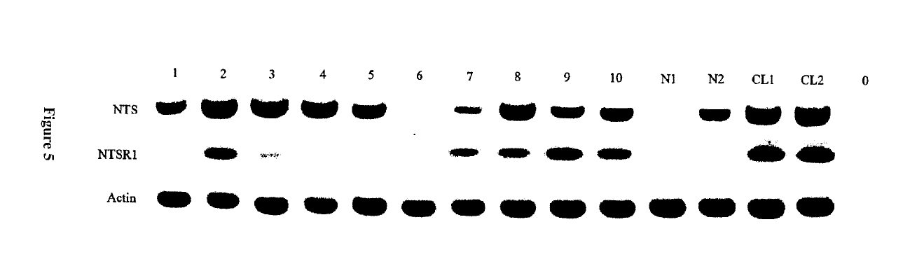

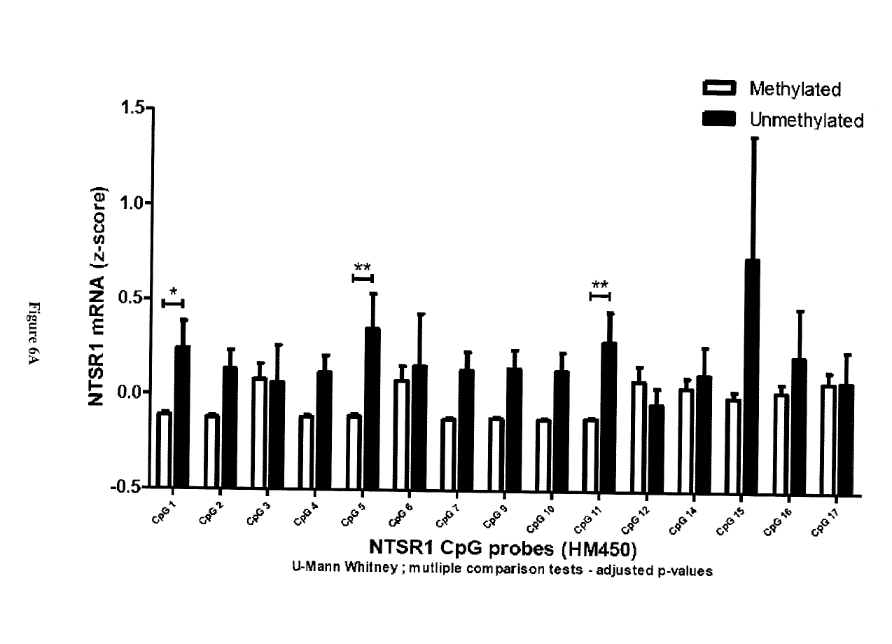

In order to evaluate if NTS was differentially expressed between benign and malignant samples, and if NTS was expressed connotatively with NTSR1, we evaluated the expression of NTS mature peptide and its precursor in endometrial carcinoma (n=18), endometrial hyperplasia (n=20) and normal endometrium (n=10) by image analysis of the global staining. In all cases a large amount of cells was positively labeled with NTS antibodies. NTS and pro NTS labeling was cytoplasmic and homogenous. Both expression was significantly higher in endometrial adenocarcinoma than in normal endometrium (p<0.0001) and endometrial hyperplasia (p<0.0001) (FIG. 4). The presence of NTSR1 ligand supported the hypothesis of the potential ligand-dependent NTSR1 activation, as the intra-cytoplasmic localization of NTSR1 would suggest. Expression of NTSR1 and NTS transcripts was confirmed in 12 samples (10 cases of adenocarcinoma and 2 cases of normal endometrium) by RT-PCR in frozen tissues (FIG. 5).

Mechanisms Involved in NTSR1 Overexpression

To understand the molecular basis causing overexpression NTSR1, we analyzed the data available in the TCGA database NTSR1. Expression was first analyzed in endometrial carcinoma according to the 4 molecular subgroups as defined by Kandoth et al.[5]. The NTSR1 mRNA level was significantly higher in the subgroup exhibiting high copy-number alterations (p<0.0001). This association remained significant when excluding the serous and mixed histological subtypes (p=0.01). No association with the MSI (microsatellite instability), POLE or low copy-number alteration molecular subgroups was found.

Focusing on copy number variation in the NTSR1 region, NTSR1 mRNA up regulation was significantly correlated with a high NTSR1 gene copy number alteration (p=0.0005), showing a higher NTSR1 mRNA z-score in cases characterized by a low-level gain (45/324) or a high-level amplification (9/324) as compared as in diploid cases (266/324). In our cohort, in order to determine if amplification could explain a high NSTR1 protein expression, we evaluated the copy number of NTSR1 gene by in-situ hybridization (20 cases). We did not find amplification in any of the 17 interpretable analyzed cases (10 cases with high protein expression, 7 cases with low protein expression) (data not shown). Additionally, in the TCGA cohort, mutations of NTSR1 were found in 4 cases (S93L; T155M; X306_splice; A260T, S181P), showing a low NTSR1 mRNA z-score (p>0.50).

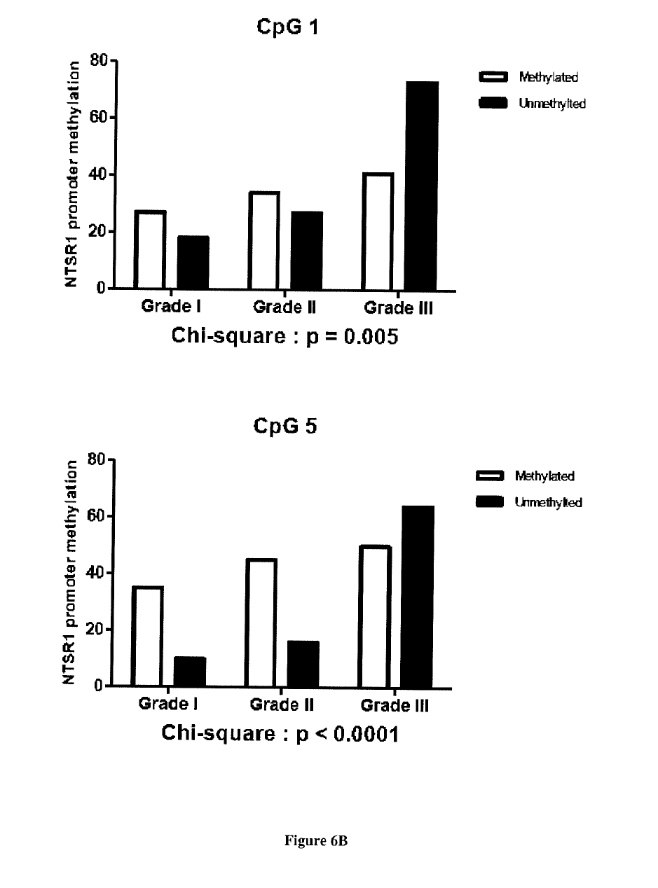

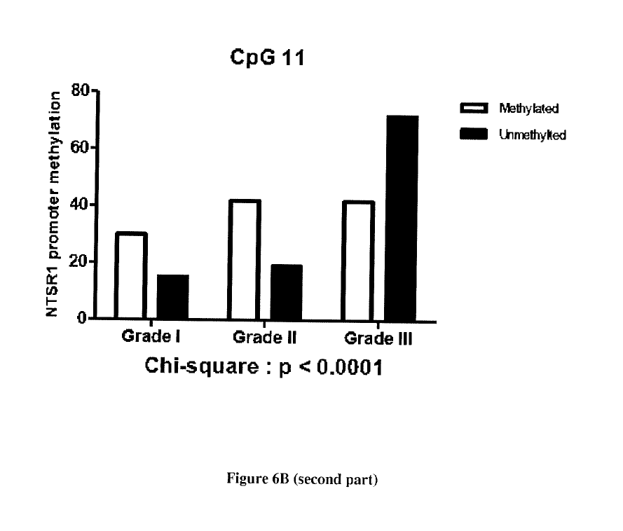

Lastly, we analyzed the correlation between NTSR1 promoter DNA methylation and expression level of mRNA NTSR1 in the TCGA UCEC cohort. Basing on the HM450 assay, data from 15 out of 17 probes covering NTSR1 CpG islands were available in 220 cases (FIG. 6A). Globally, FIG. 6A showed that most unmethylated sites were associated with a higher NTSR1 mRNA z-core. In three out of these 15 CpG (CpG 1, CpG 5 and CpG 11), loss of methylation was significantly associated with a higher NTSR1 mRNA level, suggesting that loss of methylation of NTSR1 may play a major role in its overexpression (Table 4). Additionally, when focusing on these 3 later CpG islands, we found these CpG islands were significantly unmethylated in high grade carcinomas, as compared to grade I and II tumors (CpG 1: 64% unmethylated in grade III tumors vs. 42% in grades I+II tumors, p=0.005; CpG 5: 56% vs. 25%, p<0.0001; CpG 11: 63% vs. 32%, p<0.0001) (FIG. 6B).

Discussion

NTS/NTSR1 in Endometrium

Our data demonstrates, for the first time, the contribution of NTS/NTSR1 complex in human endometrial cancer progression. We found a significant upregulation of NTSR1 expression as well as an overexpression of NTS in malignant tumors, as compared to normal endometrium. Additionally, we found a significantly higher mRNA level and cytoplasmic protein expression in more advanced tumors grade 3 vs grade 1 and 2.

Pejorative implications of the NTS/NSTR1 complex in tumor progression is supported by the inverse correlation between NTSR1 overexpression and the OS in several clinical series, such as in a selected population of lung adenocarcinomas and in invasive breast carcinomas [14, 35, 36]. We demonstrate here that NTSR1 is a marker of poor prognosis in human endometrial carcinoma, and is an independent prognosis factor in multivariate analysis, which supports its role as a contributor in endometrial cancer progression.

NTS and NTSR1 are implicated in several functions linked to the neoplastic progression, including proliferation of pancreas, prostate, colon, lung and breast cancer cells [37], the protection of breast cancer cells against apoptosis [20], and the induction of the pro-invasive potential of colon, breast and lung cancer cells [35, 36, 38]. NTS is up regulated by estradiol and may be a mediator of hormone dependent tumorigenesis, since NTS and NTSR1 were reported to be up regulated in 20% of estrogen receptors positive breast cancers [7, 8]. As this mechanism is also estrogen-dependent, similar mechanisms might also occur in endometrial tumorigenesis [39].

Little is known regarding the function of NTS in the genital tract. Early studies had reported the presence of NTS in rat uterus [26, 27]. More recently, the NTSR1 expression in mouse epididymal spermatozoa was demonstrated to be sensitive to NTS in sperm capacitation. Additionally, the presence of NTS was confirmed in epithelia of the mouse and cattle uterus and oviduct isthmus and ampulla, which correspond to the fertilization route of spermatozoa [40, 41]. NTSR1 and NTS expression has been reported in normal myometrium, and in cases of estrogen-dependent tumors, leiomyoma and leiomyosarcoma [16, 17].

Mechanisms Sustaining NTSR1 Overexpression

The presence of local NTS production in normal to endometrial carcinoma is probably an essential feature for malignant progression. We hypothesize that the contribution of NTSR1 in tumorigenesis occurred from local and sustained activation of the receptor rather than from circulating NTS, because NTS is a highly degradable peptide and its blood concentration rapidly drops after its release. In addition, it was shown that the high concentration of the long fragment NTS (LF NTS) was correlated with a higher risk of breast cancer [42]. Our immunohistochemistry experiment confirms the expression of LF NTS in endometrial tumor. Using cytoplasmic NTSR1 scoring, we found that the cytoplasmic accumulation was correlated with tumor progression and survival. On the contrary, using a membranous NTSR1 semi-quantitative score, we did not find any significant correlation between membranous staining and tumor grade or survival (data not shown). Similar results were observed in lung cancer. In adenocarcinomas subtypes, NTSR1 was localized inside of the cytoplasm, and is correlated with a worst outcome, whereas in squamous subtypes, NTSR1 was localized preferentially at the membrane and did not influence the outcome [36]. Previous studies in our laboratory also revealed, that under prolonged NTS exposure with saturating concentrations, NTSR1 did not remain at the cell membrane, but, after endocytosis, accumulated in the perinuclear recycling compartment from which it was recycled to the cell surface. This permanent NTSR1 binding recovery allowed for constant cell sensitization and led to a chronic activation of mitogen-activated protein kinases p42 and p44 [43].

Based on the UCEC cohort of TCGA, we gathered evidence suggesting that NTSR1 overexpression can be consecutive to the loss of NTSR1 promoter DNA methylation. Similarly, in colorectal tumors, low levels of methylation may contribute to the malignant potential through activation of NTSR1 [44] while NTSR1 methylation is known to be associated with lateral and noninvasive growth of colorectal tumors. Moreover in neuroendocrine tumors, DNA methylation contributes to NTSR1/2 expression patterns [45]. Copy number alterations could be an alternative mechanism leading to NTSR1 overexpression, but seemed to play a more modest role in endometrial carcinoma, since only 3% (9/324) showing high level amplification in the UCEC cohort of TCGA, and no case in our cohort, albeit with a limited number of cases tested with in situ hybridization.

Diagnostic and Therapeutic Perspectives

We propose that NTSR1 IHC could be used as a tool to select patients with the worst prognostic in order to adapt the therapy. This is a low cost, and easy technique which could be used routinely.

Moreover, NTSR1 targeting has been proposed as therapeutic tool. For example, DOTA-NT-20.3 is a promising candidate for ⁶⁸Ga-PET imaging of neurotensin receptor-positive tumors, and for therapy, with the yttrium-labeled peptide [46]. Recently, we proposed the use of labeled NTS/NTSR1 complexes to enlarge the population eligible for therapy targeting HERs tyrosine kinase inhibitor or HER2 overexpression in breast cancers [35]. The selective NTSR1 antagonist, SR48692, radio-sensitized prostate cancer cells in a dose- and time-dependent manner by increasing apoptotic cell death and decreasing clonogenic survival [47]. Due to the contribution of the neurotensinergic system in cancer progression we are able to highlight its potential as a therapeutic target [15, 48, 49]. Based on our results, therapeutic strategies targeting the abolition of NSTR1 activation could be eligible as a new alternative in the treatment of endometrial cancer, especially in advanced stage after specific study of the expression of NTSR1. Further studies are needed to support these proposals.

### CONCLUSIONS

We provided evidence for NTS/NTSR1 as a potential contributor to endometrial cancer oncogenesis and progression, through an upregulation of cytoplasmic NTSR1. We pointed out the prognostic value of NTSR1, this marker being an independent marker of poor survival. NTSR1 cytoplasmic semi-quantitative score could be easily used to evaluate the prognosis. Furthermore, identification of tumors characterized by paracrine NTS/NTSR1 signaling pathway activation could provide alternative strategies to improve the treatment. These findings support the therapeutic potential of NTS/NTSR1 inhibition or drug cellular targeting through NTSR1 in advanced stages of human endometrial cancers.

TABLE 1
Clinical and histological characteristics
of the patients (SD: standard deviation).

| | Mean ± SD or percent of patients |
|---|---|
| Clinical data (n = 100) | |
| Age in years | 67.58 ± 11.26 |
| Reproductive period in years | 38.07 ± 4.94 |
| Body Mass Index (kg/m²) | 29.32 ± 7.48 |
| Parity | 2.00 ± 1.68 |
| Hypertension | 53% |
| Diabetes mellitus | 20% |
| Breast Carcinoma | 12% |
| Menopause Hormone therapy | 20% |
| FIGO Stage | |
| Ia | 56% |
| Ib | 23% |
| II | 2% |
| IIIa | 4% |
| IIIb | 0% |
| IIIc | 4% |
| IVa | 0% |
| IVb | 11% |
| Histological type | |
| Endometrioid Adenocarcinoma | 89% |
| Serous Adenocarcinoma | 6% |
| Clear cell Adenocarcinoma | 3% |
| Mucinous Adenocarcinoma | 2% |
| Histological Grade | |
| 1 | 40% |
| 2 | 40% |
| 3 | 20% |
| Histological Characteristics | |
| Myometrial invasion ≥ 50% | 41% |
| Neoplastic vascular emboli | 19% |
| Adjuvant therapy | |
| Brachytherapy | 75% |
| Radiotherapy | 23% |
| Chemotherapy | 6% |

TABLE 2
Sequence of PCR primers

| Strand Name | Oligonucleotide sequence (5′→3′) |
|---|---|
| Actin (forward) | ACC AAC TGG GAC GAC ATG GAG AAA |
| Actin (reverse) | GGG ATA GCA CAG CCT GGA TAG CA |
| NTS (forward) | GCT TTA GCT TGG AAG CAA TGT T |
| NTS (reverse) | TCA TAC AGC TGC CGT TTC AG |
| NTSR1 (forward) | GGC GCC TCAT GTT CTG CTA |
| NTSR1 (reverse) | GTG CGT TGG TCA CCA TGT AGA |

TABLE 3
NTSR1 expression according to the histologic
type and histological grade.

| | NTSR1 Global | | NTSR1 | |
|---|---|---|---|---|
| | staining score | | Cytoplasmic | |
| Histological type | (×10⁵) | p | Score | p |
| Endometrial adeno- | 75.83 ± 15.90 | p < | 49 ± 5 | p < |
| carcinoma (n = 100) | | 0.001 | | 0.001 |
| Benign endometrium | 5.53 ± 0.66 | | 15 ± 2 | |
| (n = 66) | | | | |
| Hyperplasia (n = 35) | 5.86 ± 1.01 | | 14 ± 3 | |
| Polyp (n = 14) | 4.33 ± 1.24 | | 14 ± 4 | |
| Normal endometrium | 5.86 ± 1.10 | | 18 ± 6 | |
| (n = 17) | | | | |
| Histological Grade | | p = | | p = |
| | | 0.01 | | 0.004 |
| 1 (n = 40) | 29.42 ± 4.33 | | 30 ± 5 | |
| 2 (n = 40) | 67.29 ± 18.92 | | 45 ± 7 | |
| 3 (n = 20) | 185.72 ± 64.56 | | 94 ± 13 | |

Kruskal-Wallis test; histological type: adenocarcinoma vs. benign samples; grade 3 vs. grade 1 + grade 2. Results are expressed as mean value ± standard error mean

TABLE 4
Correlation between the NTSR1 promoter methylation of and NTSR1 mRNA expression
in the uterine corpus endometrial carcinoma cohort of TCGA (HM450; n = 220).

| | | | | | NTSR1 mRNA average Z-Score | | Adjusted |
|---|---|---|---|---|---|---|---|
| CpG | CpG island | Genomic position | Methylated cases | | Unmethylated | Methylated | p-value |
| CpG 1 | cg00254133 | 61340542 | 46.8% | (103/220) | 0.237 | −0.109 | 0.047 |
| CpG 2 | cg01539036 | 61340116 | 21.8% | (48/220) | 0.132 | −0.123 | 0.642 |
| CpG 3 | cg02216247 | 61342017 | 91.4% | (201/220) | 0.062 | 0.078 | 1 |
| CpG 4 | cg02658437 | 61339804 | 15.9% | (35/220) | 0.113 | −0.119 | 1 |
| CpG 5 | cg03567830 | 61339871 | 59.1% | (130/220) | 0.350 | −0.113 | 0.002 |
| CpG 6 | cg04834228 | 61392703 | 99.1% | (218/220) | 0.153 | 0.076 | 1 |
| CpG 7 | cg05295038 | 61340107 | 21.8% | (48/220) | 0.133 | −0.126 | 0.278 |
| CpG 8 | cg07938252 | 61340827 | 100% | (220/220) | NA | 0.076 | NA |
| CpG 9 | cg08678514 | 61340340 | 25% | (55/220) | 0.142 | −0.120 | 1 |
| CpG 10 | cg09893588 | 61340109 | 21.4% | (47/220) | 0.131 | −0.125 | 0.351 |
| CpG 11 | cg11877251 | 61339875 | 51.8 | (114/220) | 0.287 | −0.119 | 0.002 |
| CpG 12 | cg12910641 | 61343222 | 96.8% | (213/220) | −0.041 | 0.080 | 1 |
| CpG 13 | cg13213889 | 61371658 | NA | | NA | NA | NA |
| CpG 14 | cg13292703 | 61371808 | 53.2% | (117/220) | 0.113 | 0.044 | 1 |
| CpG 15 | cg14871138 | 61340885 | 89.1% | (196/220) | 0.736 | −0.004 | 1 |
| CpG 16 | cg16430535 | 61342164 | 72.7% | (160/220) | 0.211 | 0.026 | 1 |
| CpG 17 | cg24235518 | 61371425 | 57.7% | (127/220) | 0.078 | 0.075 | 1 |

U-Mann Whitney test, Bonferroni adjustment of p-values for multiple comparisons

### REFERENCES

Throughout this application, various references describe the state of the art to which this invention pertains. The disclosures of these references are hereby incorporated by reference into the present disclosure.
1. Siegel R, Ma J, Zou Z, Jemal A: Cancer statistics, 2014, CA Cancer J Clin 2014, 64:9-29
2. Fleming G F, Brunetto V L, Cella D, Look K Y, Reid G C, Munkarah A R, Kline R, Burger R A, Goodman A, Burks R T: Phase III trial of doxorubicin plus cisplatin with or without paclitaxel plus filgrastim in advanced endometrial carcinoma: a Gynecologic Oncology Group Study, J Clin Oncol 2004, 22:2159-2166
3. Thigpen J T, Brady M F, Homesley H D, Malfetano J, DuBeshter B, Burger R A, Liao S: Phase III trial of doxorubicin with or without cisplatin in advanced endometrial carcinoma: a gynecologic oncology group study, J Clin Oncol 2004, 22:3902-3908
4. van Wijk F H, Aapro M S, Bolis G, Chevallier B, van der Burg M E, Poveda A, de Oliveira C F, Tumolo S, Scotto di Palumbo V, Piccart M, Franchi M, Zanaboni F, Lacave A J, Fontanelli R, Favalli G, Zola P, Guastalla J P, Rosso R, Marth C, Nooij M, Presti M, Scarabelli C, Splinter T A, Ploch E, Beex L V, ten Bokkel Huinink W, Forni M, Melpignano M, Blake P, Kerbrat P, Mendiola C, Cervantes A, Goupil A, Harper P G, Madronal C, Namer M, Scarfone G, Stoot J E, Teodorovic I, Coens C, Vergote I, Vermorken J B: Doxorubicin versus doxorubicin and cisplatin in endometrial carcinoma: definitive results of a randomised study (55872) by the EORTC Gynaecological Cancer Group, Ann Oncol 2003, 14:441-448
5. Kandoth C, Schultz N, Cherniack A D, Akbani R, Liu Y, Shen H, Robertson A G, Pashtan I, Shen R, Benz C C, Yau C, Laird P W, Ding L, Zhang W, Mills G B, Kucherlapati R, Mardis E R, Levine D A: Integrated genomic characterization of endometrial carcinoma, Nature 2013, 497:67-73
6. Sun H, Yan L, Tu R, Zhang Y, Ma L, Tang W, Li L, Chen W, Zhan C, Zang R: Expression Profiles of Endometrial Carcinoma by Integrative Analysis of TCGA Data, Gynecol Obstet Invest 2016,
7. Dupouy S, Viardot-Foucault V, Alifano M, Souaze F, Plu-Bureau G, Chaouat M, Lavaur A, Hugol D, Gespach C, Gompel A, Forgez P: The neurotensin receptor-1 pathway contributes to human ductal breast cancer progression, PLoS One 2009, 4:e4223
8. Souaze F, Dupouy S, Viardot-Foucault V, Bruyneel E, Attoub S, Gespach C, Gompel A, Forgez P: Expression of neurotensin and NT1 receptor in human breast cancer: a potential role in tumor progression, Cancer Res 2006, 66:6243-6249
9. Reinecke M: Neurotensin. Immunohistochemical localization in central and peripheral nervous system and in endocrine cells and its functional role as neurotransmitter and endocrine hormone, Prog Histochem Cytochem 1985, 16:1-172
10. Andersson S, Rosell S, Hjelmquist U, Chang D, Folkers K: Inhibition of gastric and intestinal motor activity in dogs by (Gln4) neurotensin, Acta Physiol Scand 1977, 100:231-235
11. Armstrong M J, Parker M C, Ferris C F, Leeman S E: Neurotensin stimulates [3H]oleic acid translocation across rat small intestine, Am J Physiol 1986, 251:G823-829
12. Baca I, Feurle G E, Schwab A, Mittmann U, Knauf W, Lehnert T: Effect of neurotensin on exocrine pancreatic secretion in dogs, Digestion 1982, 23:174-183
13. Mazella J: Sortilin/neurotensin receptor-3: a new tool to investigate neurotensin signaling and cellular trafficking?, Cell Signal 2001, 13:1-6
14. Alifano M, Souaze F, Dupouy S, Camilleri-Broet S, Younes M, Ahmed-Zaid S M, Takahashi T, Cancellieri A, Damiani S, Boaron M, Broet P, Miller L D, Gespach C, Regnard J F, Forgez P: Neurotensin receptor 1 determines the outcome of non-small cell lung cancer, Clin Cancer Res 2010, 16:4401-4410
15. Dupouy S, Mourra N, Doan V K, Gompel A, Alifano M, Forgez P: The potential use of the neurotensin high affinity receptor 1 as a biomarker for cancer progression and as a component of personalized medicine in selective cancers, Biochimie 2011, 93:1369-1378
16. Rodriguez Y, Almeida T A, Valladares F, Baez D, Montes de Oca F, Garcia C, Dorta I, Hernandez M, Reyes R, Bello A R: Neurotensin and neurotensin receptor 1 expression in human myometrium and uterine leiomyomas, Biol Reprod 2010, 83:641-647
17. Rodriguez Y, Baez D, de Oca F M, Garcia C, Dorta I, Reyes R, Valladares F, Almeida T A, Bello A R: Comparative analysis of the ERalpha/ERbeta ratio and neurotensin and its high-affinity receptor in myometrium, uterine leiomyoma, atypical leiomyoma, and leiomyosarcoma, Int J Gynecol Pathol 2011, 30:354-363
18. Sakumoto R, Hayashi K G, Saito S, Kanahara H, Kizaki K, Iga K: Comparison of the global gene expression profiles in the bovine endometrium between summer and autumn, J Reprod Dev 2015, 61:297-303
19. Elek J, Pinzon W, Park K H, Narayanan R: Relevant genomics of neurotensin receptor in cancer, Anticancer Res 2000, 20:53-58
20. Somai S, Gompel A, Rostene W, Forgez P: Neurotensin counteracts apoptosis in breast cancer cells, Biochem Biophys Res Commun 2002, 295:482-488
21. Wang L, Friess H, Zhu Z, Graber H, Zimmermann A, Korc M, Reubi J C, Buehler M W: Neurotensin receptor-1 mRNA analysis in normal pancreas and pancreatic disease, Clin Cancer Res 2000, 6:566-571
22. Soler M, Chatenoud L, Negri E, Parazzini F, Franceschi S, la Vecchia C: Hypertension and hormone-related neoplasms in women, Hypertension 1999, 34:320-325
23. Soliman P T, Wu D, Tortolero-Luna G, Schmeler K M, Slomovitz B M, Bray M S, Gershenson D M, Lu K H: Association between adiponectin, insulin resistance, and endometrial cancer, Cancer 2006, 106:2376-2381
24. Alexander M J: Estrogen-regulated synthesis of neurotensin in neurosecretory cells of the hypothalamic arcuate nucleus in the female rat, Endocrinology 1993, 133:1809-1816
25. Alexander M J, Leeman S E: Estrogen-inducible neurotensin immunoreactivity in the preoptic area of the female rat, J Comp Neurol 1994, 345:496-509
26. Mistry A, Vijayan E: Neurotensin enhances estradiol induced DNA synthesis in immature rat uterus, Life Sci 1985, 36:2063-2067
27. Pettibone D J, Totaro J A: High affinity binding of [3H]neurotensin of rat uterus, Peptides 1987, 8:1169-1172
28. Watters J J, Dorsa D M: Transcriptional effects of estrogen on neuronal neurotensin gene expression involve cAMP/protein kinase A-dependent signaling mechanisms, J Neurosci 1998, 18:6672-6680
29. Gao J, Aksoy B A, Dogrusoz U, Dresdner G, Gross B, Sumer S O, Sun Y, Jacobsen A, Sinha R, Larsson E, Cerami E, Sander C, Schultz N: Integrative analysis of complex cancer genomics and clinical profiles using the cBioPortal, Sci Signal 2013, 6: p11
30. Cerami E, Gao J, Dogrusoz U, Gross B E, Sumer S O, Aksoy B A, Jacobsen A, Byrne C J, Heuer M L, Larsson E, Antipin Y, Reva B, Goldberg A P, Sander C, Schultz N: The cBio cancer genomics portal: an open platform for exploring multidimensional cancer genomics data, Cancer Discov 2012, 2:401-404
31. Kurman R J, Carcangiu M L, Herrington C S, Young R H: World Health Organisation Classification of Tumours of Female Reproductive Organs. Edited by (IARC) IAfRoC. Lyon, IARC Press, 2014,
32. Petru E, Luck H J, Stuart G, Gaffney D, Millan D, Vergote I: Gynecologic Cancer Intergroup (GCIG) proposals for changes of the current FIGO staging system, Eur J Obstet Gynecol Reprod Biol 2009, 143:69-74
33. Giorgi R R, Chile T, Bello A R, Reyes R, Fortes M A, Machado M C, Cescato V A, Musolino N R, Bronstein M D, Giannella-Neto D, Correa-Giannella M L: Expression of neurotensin and its receptors in pituitary adenomas, J Neuroendocrinol 2008, 20:1052-1057
34. Taylor R M, Severns V, Brown D C, Bisoffi M, Sillerud L O: Prostate cancer targeting motifs: expression of alphanu beta3, neurotensin receptor 1, prostate specific membrane antigen, and prostate stem cell antigen in human prostate cancer cell lines and xenografts, Prostate 2012, 72:523-532
35. Dupouy S, Doan V K, Wu Z, Mourra N, Liu J, De Wever O, Llorca F P, Cayre A, Kouchkar A, Gompel A, Forgez P: Activation of EGFR, HER2 and HER3 by neurotensin/neurotensin receptor 1 renders breast tumors aggressive yet highly responsive to lapatinib and metformin in mice, Oncotarget 2014, 5:8235-8251
36. Younes M, Wu Z, Dupouy S, Lupo A M, Mourra N, Takahashi T, Flejou J F, Tredaniel J, Regnard J F, Damotte D, Alifano M, Forgez P: Neurotensin (NTS) and its receptor (NTSR1) causes EGFR, HER2 and HER3 over-expression and their autocrine/paracrine activation in lung tumors, confirming responsiveness to erlotinib, Oncotarget 2014, 5:8252-8269
37. Evers B M: Neurotensin and growth of normal and neoplastic tissues, Peptides 2006, 27:2424-2433
38. Souaze F, Viardot-Foucault V, Roullet N, Toy-Miou-Leong M, Gompel A, Bruyneel E, Comperat E, Faux M C, Mareel M, Rostene W, Flejou J F, Gespach C, Forgez P: Neurotensin receptor 1 gene activation by the Tcf/beta-catenin pathway is an early event in human colonic adenomas, Carcinogenesis 2006, 27:708-716
39. Shapiro S, Kelly J P, Rosenberg L, Kaufman D W, Helmrich S P, Rosenshein N B, Lewis J L, Jr., Knapp R C, Stolley P D, Schottenfeld D: Risk of localized and widespread endometrial cancer in relation to recent and discontinued use of conjugated estrogens, N Engl J Med 1985, 313:969-972
40. Umezu K, Hiradate Y, Oikawa T, Ishiguro H, Numabe T, Hara K, Tanemura K: Exogenous neurotensin modulates sperm function in Japanese Black cattle, J Reprod Dev 2016,
41. Hiradate Y, Inoue H, Kobayashi N, Shirakata Y, Suzuki Y, Gotoh A, Roh S G, Uchida T, Katoh K, Yoshida M, Sato E, Tanemura K: Neurotensin enhances sperm capacitation and acrosome reaction in mice, Biol Reprod 2014, 91:53
42. Melander O, Maisel A S, Almgren P, Manjer J, Belting M, Hedblad B, Engstrom G, Kilger U, Nilsson P, Bergmann A, Orho-Melander M: Plasma proneurotensin and incidence of diabetes, cardiovascular disease, breast cancer, and mortality, Jama 2012, 308:1469-1475
43. Toy-Miou-Leong M, Cortes C L, Beaudet A, Rostene W, Forgez P: Receptor trafficking via the perinuclear recycling compartment accompanied by cell division is necessary for permanent neurotensin cell sensitization and leads to chronic mitogen-activated protein kinase activation, J Biol Chem 2004, 279:12636-12646
44. Kamimae S, Yamamoto E, Kai M, Niinuma T, Yamano H O, Nojima M, Yoshikawa K, Kimura T, Takagi R, Harada E, Harada T, Maruyama R, Sasaki Y, Tokino T, Shinomura Y, Sugai T, Imai K, Suzuki H: Epigenetic silencing of NTSR1 is associated with lateral and noninvasive growth of colorectal tumors, Oncotarget 2015, 6:29975-29990
45. Kim J T, Li J, Song J, Lee E Y, Weiss H L, Townsend C M, Jr., Evers B M: Differential expression and tumorigenic function of neurotensin receptor 1 in neuroendocrine tumor cells, Oncotarget 2015, 6:26960-26970
46. Alshoukr F, Prignon A, Brans L, Jallane A, Mendes S, Talbot J N, Tourwe D, Barbet J, Gruaz-Guyon A: Novel DOTA-neurotensin analogues for 111In scintigraphy and 68Ga PET imaging of neurotensin receptor-positive tumors, Bioconjug Chem 2011, 22:1374-1385
47. Valerie N C, Casarez E V, Dasilva J O, Dunlap-Brown M E, Parsons S J, Amorino G P, Dziegielewski J: Inhibition of neurotensin receptor 1 selectively sensitizes prostate cancer to ionizing radiation, Cancer Res 2011, 71:6817-6826
48. Morgat C, Mishra A K, Varshney R, Allard M, Fernandez P, Hindie E: Targeting neuropeptide receptors for cancer imaging and therapy: perspectives with bombesin, neurotensin, and neuropeptide-Y receptors, J Nucl Med 2014, 55:1650-1657
49. Wu Z, Martinez-Fong D, Tredaniel J, Forgez P: Neurotensin and its high affinity receptor 1 as a potential pharmacological target in cancer therapy, Front Endocrinol (Lausanne) 2012, 3:184

1. A method for predicting the survival time of a subject suffering from an endometrial adenocarcinoma comprising i) determining the expression level of NTSR1 expression in a tumor tissue sample obtained from the subject, ii) comparing the expression level determined at step i) with its predetermined reference value and ii) concluding that the subject will have a long survival time when the expression level of NTSR1 is lower than its predetermined reference value or concluding that the subject will have a short survival time when the expression level of NTSR1 is higher than its predetermined reference value.

2. The method of claim 1 wherein the expression level of NTSR1 is determined at nucleic acid level.

3. The method of claim 1 wherein the expression level of NTSR1 is determined at the protein level.

4. The method of claim 1 wherein the expression level of NTSR1 is determined by immunohistochemistry.

5. The method of claim 1 wherein the NTSR1 cytoplasmic expression level is determined.

6. A method of treating endometrial cancer in a subject in need thereof comprising administering to the subject a therapeutically effective amount of an inhibitor of NTSR1 activation or expression.

7. The method of claim 6 wherein the inhibitor of NTSR1 activation is an antibody against NTS or a fragment thereof which binds to NTS, or an antibody against the NTSR1 or a fragment thereof which binds to the NTSR1.

8. The method of claim 6 wherein the inhibitor of NTSR1 activation is an anti-NTSR1 monoclonal antibody-drug conjugate.

9. The method of claim 6 wherein the inhibitor of NTSR1 expression is small inhibitory RNAs.

10. The method of claim 6 wherein the inhibitor of NTSR1 expression is ribozymes.
